(19) United States
(12) Patent Application Publication
(10) Pub. No.: US 2026/0165863 A1
(43) Pub. Date: Jun. 18, 2026

(54) STENT DELIVERY DEPLOYMENT ACCURACY AIDS

(71) Applicant: BOSTON SCIENTIFIC SCIMED, INC., Maple Grove, MN (US)

(72) Inventors: GARY GILMARTIN, Foxford (IE); FIONN STAPLETON, Oranmore (IE); CELINE GLYNN, Galway (IE); DANIEL TUCK, Galway (IE); KEITH O'REILLY, Galway (IE); DAVID GILES, Tuam (IE); HARRY MULLEADY, Kinvara (IE); THOMAS MARTIN KEATING, Tuam (IE); SHANE O'KEEFFE, Tuam (IE); AMANDA MATASHU, Sligo (IE); JOHN THOMAS O'DRISCOLL, Galway (IE)

(73) Assignee: BOSTON SCIENTIFIC SCIMED, INC., Maple Grove, MN (US)

(21) Appl. No.: 19/420,166

(22) Filed: Dec. 15, 2025

Related U.S. Application Data
(60) Provisional application No. 63/734,451, filed on Dec. 16, 2024.

(51) Int. Cl. A61F 2/966; A61F 2/95

(52) U.S. Cl. CPC A61F 2/966; A61F 2/9517

(57) ABSTRACT

This disclosure addresses deployment accuracy challenges in co-axial stent delivery systems by incorporating various ancillary devices: a tubular curved track that constrains catheter movement, a handle clamp that secures the proximal handle position, and/or a stiffening support shaft that can be inserted into a lumen of the catheter. The tubular curved track features a loop body with a channel along its length to receive and stabilize the catheter's proximal portion. The handle clamp includes a cavity and clamping mechanism to secure the proximal handle to a support structure. The stiffening support shaft can be inserted to various depths to prevent unwanted catheter movement. These ancillary devices can be used individually or in combination to enhance stent deployment accuracy by reducing unintended movements during the deployment procedure.

### CROSS REFERENCE TO RELATED APPLICATIONS

The present application claims the benefit of U.S. Provisional Patent Application Ser. No. 63/734,451, filed Dec. 16, 2024, the disclosure of which is incorporated herein by reference.

### TECHNICAL FIELD

The present disclosure generally relates to deployment of self-expanding stents in co-axial stent deployment systems. More specifically, the present disclosure is directed towards improving deployment accuracy of the placement of the stent through the use of an ancillary device.

### BACKGROUND

Many co-axial stent deployment systems are operated through a bi-handle system in which a first handle is actuated relative to a second handle of the deployment system. For example, to deploy the self-expanding stent, the user or multiple users retract one of the handles while attempting to hold the other handle steady.

However, when the handle is actuated, inadvertent movement of the other handle and/or the catheter shaft extending from the other handle may inadvertently move the stent within the body lumen or other target location, affecting deployment accuracy of the stent relative to the desired deployment location with a patient's anatomy. Thus, to reduce or eliminate inaccuracies in stent deployment, a need for ancillary devices to reduce or eliminate inadvertent movements to the co-axial deployment system exists.

### BRIEF SUMMARY

A first example is a stent delivery catheter system. The stent delivery system includes a stent and a catheter shaft having a distal portion and a proximal portion. The catheter shaft includes an inner shaft having a proximal end and a distal end, and an outer tubular shaft having a proximal end and a distal end. The outer tubular shaft is slidably disposed over the inner shaft. The outer tubular shaft is configured to hold the stent in a constrained position proximate the distal end of the outer tubular shaft. The stent is configured to be deployed from the outer tubular shaft when the outer tubular shaft is retracted proximally relative to the stent. A handle assembly is provided at a proximal end of the catheter shaft. The handle assembly includes a proximal handle coupled to the proximal end of the inner shaft and a distal handle coupled to the proximal end of the outer shaft. The outer shaft is configured to retract proximally together with proximal retraction of the distal handle while the proximal handle is held in a stable position to deploy the stent. The system also includes an ancillary device including a loop body defining a loop track. The loop body has channel extending along a length of the loop body permitting the proximal portion of the catheter shaft to be laterally inserted into the loop track. The loop body is configured to constrain movement of the proximal portion of the catheter during deployment of the stent.

Alternatively or additionally to any of the examples herein, the loop track ancillary device is a loop track extending along a length of a loop body.

Alternatively or additionally to any of the examples herein, the loop track has a channel extending along a length of the loop body, permitting the proximal portion of the catheter shaft to be laterally inserted into the loop track and constrain the movement of the proximal portion of the catheter during deployment.

Alternatively or additionally to any of the examples herein, the channel extends along the length of the loop body, permitting lateral access to the loop track through a sidewall of the loop body.

Alternatively or additionally to any of the examples herein, the loop body has an arced region extending in an arcuate path.

Alternatively or additionally to any of the examples herein, the arched region curves through an arc of 180 degrees or more.

Alternatively or additionally to any of the examples herein, opposing end portions of the loop body extending from the arced region converge toward one another without crossing one another.

Alternatively or additionally to any of the examples herein, the channel opens out to an inner radius of the arced region.

Alternatively or additionally to any of the examples herein, the loop body crosses over itself at a crossover location.

Alternatively or additionally to any of the examples herein, the opposing end portions of the loop body extending from the arced region converge toward one another without crossing one another.

Alternatively or additionally to any of the examples herein, the channel of the loop body extends an entire length of the loop body.

Alternatively or additionally to any of the examples herein, the distal end of the loop track is configured to engage an access port of a working channel of an endoscope.

Another example is a stent delivery catheter system. The stent delivery catheter system includes a stent delivery system including a stent and a catheter shaft including a distal portion and a proximal portion. The catheter shaft includes an inner shaft including a proximal end and a distal end, and an outer tubular shaft including a proximal end and a distal end. The outer tubular shaft is slidably disposed over the inner shaft. The outer tubular shaft is configured to hold the stent in a constrained position proximate the distal end of the outer tubular shaft. The stent is configured to be deployed from the outer tubular shaft when the outer tubular shaft is retracted proximally relative to the stent. A handle assembly is provided at a proximal end of the catheter shaft. The handle assembly includes a proximal handle coupled to the proximal end of the inner shaft and a distal handle coupled to the proximal end of the outer shaft. The outer shaft is configured to retract proximally together with proximal retraction of the distal handle while the proximal handle is held in a stable position to deploy the stent. The system also includes an ancillary device including a handle clamp configured to hold the proximal handle in a stable position. The handle clamp includes a cavity configured to receive the proximal handle and securely fit the proximal handle to the handle clamp and a clamping mechanism configured to clamp the handle clamp to a support structure.

Alternatively or additionally to any of the examples herein, wherein the cavity is configured to receive the proximal handle therein without use of any tools.

Alternatively or additionally to any of the examples herein, the ancillary device further includes a loop body defining a curved loop track. The curved loop track opens out through a sidewall of the loop body along a length of the loop body. The curved loop track is configured to receive the proximal portion of the catheter laterally therein through a slot in the sidewall of the loop body. The loop body is configured to constrain the proximal portion of the catheter shaft during deployment of the stent.

Another example of a stent delivery system includes a stent and a catheter shaft including a distal portion and a proximal portion. The catheter shaft includes an inner shaft, having a proximal end and a distal end, and an outer tubular shaft having a proximal end and a distal end. The outer tubular shaft is slidably disposed over the inner shaft. The outer tubular shaft is configured to hold the stent in a constrained position proximate the distal end of the outer tubular shaft. The stent is configured to be deployed from the outer tubular shaft when the outer tubular shaft is retracted proximally relative to the stent. A handle assembly is provided at a proximal end of the catheter shaft. The handle assembly includes a proximal handle coupled to the proximal end of the inner shaft and a distal handle coupled to the proximal end of the outer shaft. The outer shaft is configured to retract proximally together with proximal retraction of the distal handle while the proximal handle is held in a stable position to deploy the stent. The system also includes an ancillary device including a stiffening support shaft configured to be removably inserted into a lumen of the inner shaft through an opening at a proximal end of the proximal handle.

Alternatively or additionally to any of the examples herein, the stiffening support shaft has a length less than the entire length of the catheter shaft.

Alternatively or additionally to any of the examples herein, the stiffening support shaft includes at least one visual indicator.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A is a side view of an exemplary stent delivery system.

FIG. 1B illustrates another side view of the exemplary stent delivery system of FIG. 1A.

FIG. 2A illustrates an ancillary device in accordance with one embodiment of the subject matter disclosed herein.

FIG. 2B is an enlarged view of a portion of the ancillary device of FIG. 2A.

FIG. 2C illustrates the ancillary device of FIG. 2A in combination with the stent delivery system.

FIG. 3A illustrates an ancillary device in accordance with another embodiment of the subject matter disclosed herein.

FIG. 3B illustrates the ancillary device of FIG. 3A in combination with the stent delivery system.

FIG. 4 illustrates the stent delivery system with multiple ancillary devices in accordance with an embodiment of the subject matter disclosed herein.

FIG. 5A illustrates an ancillary device in accordance with another embodiment of the subject matter disclosed herein.

FIG. 5B is schematic side view of the ancillary device of FIG. 5A in combination with the stent delivery system.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

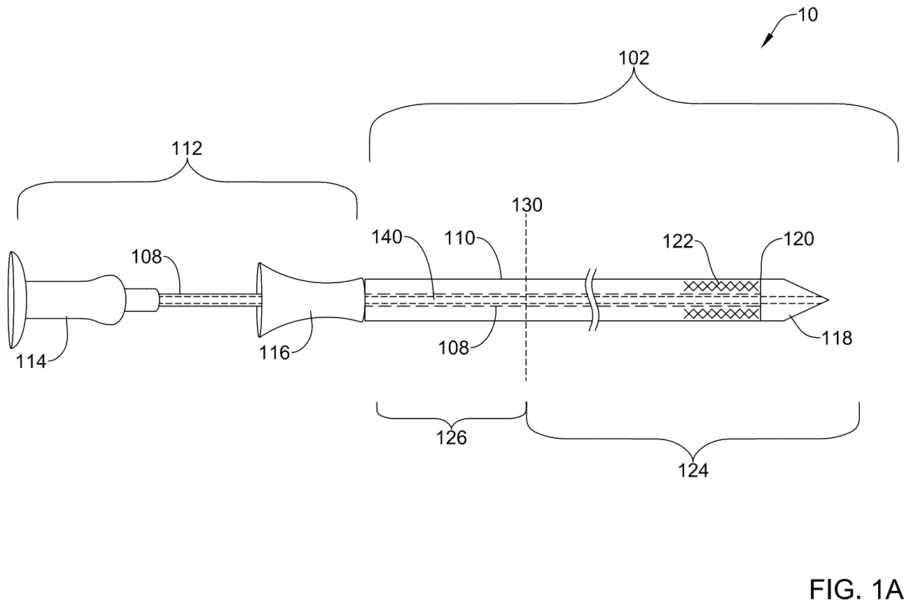

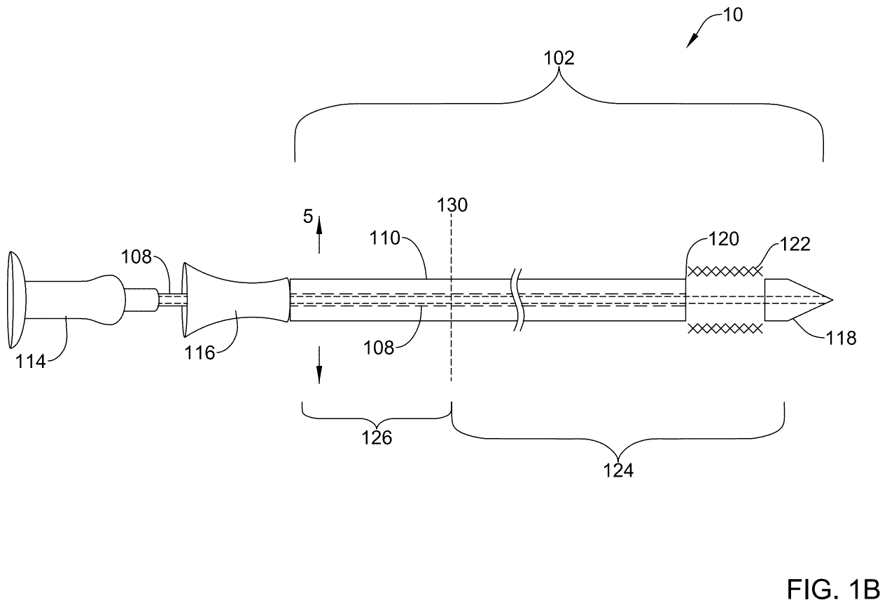

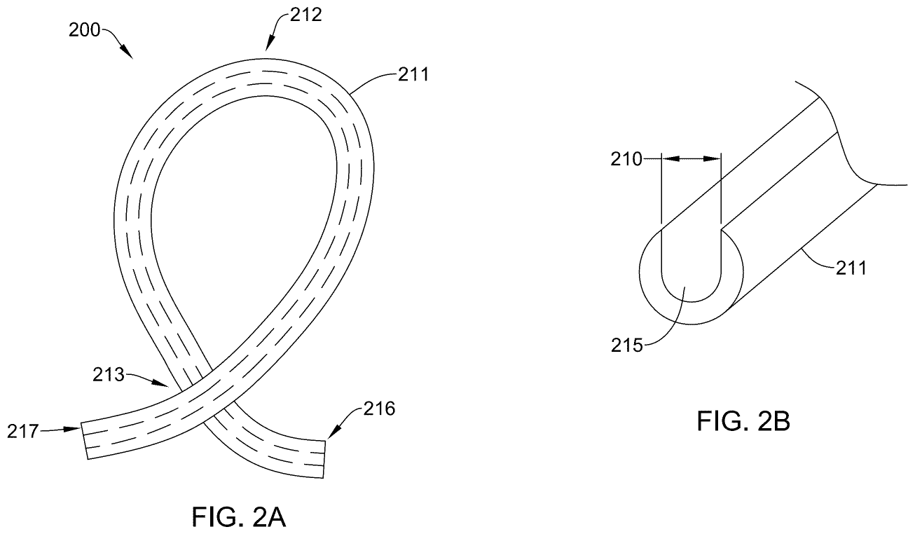

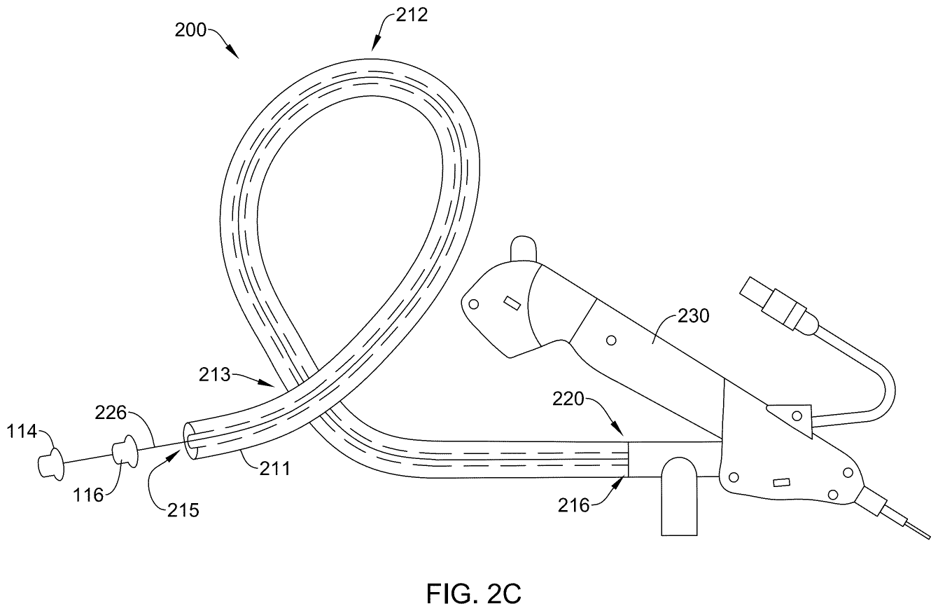

FIG. 1A shows a side view of a portion of a stent delivery system 10, shown without an ancillary device. Stent delivery catheter system 10 includes a catheter shaft 102 to deploy and deliver a self-expanding stent 122 in a bodily lumen. The distal portion 124 of catheter shaft 102 is configured to be inserted through the working channel of an endoscope (see FIG. 2C) while the remaining proximal portion 126 of the catheter shaft 102 remains proximal to the endoscope and extends proximally outside the body of a patient. The dashed demarcating line 130 of FIG. 1 represents the location that the catheter shaft 102 enters the working channel of an endoscope via an access port of the endoscope, delineating the distal portion 124 of the catheter shaft 102 inserted through the endoscope and the proximal portion 126 of the catheter shaft 102 remaining exterior of the endoscope. It is understood that the position of the demarcating line 130 along the catheter shaft 102 may vary depending on the length of the elongate shaft of the endoscope and the amount the catheter shaft 102 of the stent delivery system 10 is advanced through the working channel beyond the distal end of the elongate shaft of the endoscope.

The catheter shaft 102 may include an inner shaft 108 and an outer tubular shaft 110, each with a distal and proximal end. The outer tubular shaft 110 is slidably disposed over and surrounds the inner shaft 108, with the inner shaft 108 extending through a lumen of the outer tubular shaft 110. The distal end of the inner shaft 108 is secured to a distal tip 118 (e.g., nosecone), and the distal end 120 of the outer tubular shaft 110 may engage the distal tip 118. In a delivery configuration, a stent 122 surrounds a distal end region of the inner shaft and positioned in a radially constrained configuration within the lumen of the outer tubular shaft 110 such that the stent 222 is compressably constrained by the outer tubular shaft 110 at the distal end of catheter 102.

To operate, the stent delivery catheter system 10 includes a handle assembly 112 positioned at the proximal end of the catheter shaft 102. The handle assembly 112 includes a first, distal handle 116 and a second, proximal handle 114. The proximal handle 114 is connected to the proximal end of inner shaft 108 and the distal handle 116 is connected to the proximal end of outer shaft 110. Once the stent 122 has been advanced within the catheter shaft 102 to the target site within a body lumen, the user, typically a physician or medical technician, may operate the stent delivery catheter system 10 to deploy the stent 122 at the target site. To deploy the stent, the user actuates the distal handle 116 proximally toward the proximal handle 114, to cause the outer tubular shaft 110 to slide proximally relative to the inner shaft 108 to uncover the stent 122, as shown in FIG. 1B. For example, the user may attempt to hold the proximal handle 114 steady with one hand while retracting the distal handle 116 with the other hand. In other examples, two users may operate the stent delivery catheter system 10, each user holding one of the handles. When the distal handle 116 is proximally retracted, the outer shaft 110 is proximally retracted relative to the inner shaft 108, exposing and releasing the stent 122 from its constrained position for placement at the target site.

In some embodiments, catheter shaft 102 (e.g., the inner shaft 108) includes a lumen 140 configured to receive a guidewire. In an over-the wire stent delivery system, guidewire lumen 140 extends longitudinally from the proximal end of the proximal handle 114 to the distal end of distal tip 118. In this type of stent delivery catheter system, the user may initially place the guidewire in the patient, then slide the catheter shaft 102 over the guidewire to place the catheter shaft 102, with the stent 122 loaded therein, at the target site. In a rapid exchange stent delivery system (not illustrated), the guidewire lumen 140 extends between a proximal guidewire opening of inner shaft 108 distal of the handle assembly 112 to the distal guidewire opening at the distal end of the distal tip 118, such that the guidewire extends along the outside of the proximal portion 126 of the catheter 102 once placed.

To ensure accurate deployment of the stent 122 and minimize trauma to the patient, it is important that the only movement outside the endoscope be of the distal handle 116 moving the outer tubular shaft 110 proximally. Other movement may cause inaccuracies in deployment of the stent 122. As an example, the proximal portion 124 of the catheter 102 (the portion external of the working channel of the endoscope) may be inadvertently moved, such as moved laterally or transverse to the longitudinal axis of the catheter shaft 102 (shown by arrows 5 in FIG. 1B), in which case that movement may consequently transfer to the inner shaft 108, causing the inner shaft 108, and accordingly the distal tip 118 and the stent 122, to move proximally or distally relative to the distal end of the elongate shaft of the endoscope, and thus inadvertently shift the stent 122 from its desired deployment location. As a result, the stent 122 is released off-target.

Relatedly, to ensure accurate deployment of the stent 122, the proximal handle 114 must be held steady during the proximal retraction of the distal handle 116 to withdraw the outer tubular member 110 to uncover the stent 122. Typically, when a user retracts the distal handle 116, the user presses the proximal handle to their chest as a stop to steady the stent delivery catheter system 10. However, even slight unintended movements, for example, the movement of the chest during inhalation or exhalation, may drive the stent 122 and the distal tip 118 proximally or distally, and thus inadvertently shift the stent 122 from its desired deployment location. As a result, the stent 122 is released off-target.

In some examples, the stent delivery catheter system 10 is configured to be bi-directional in order to reconstrain the stent 122 after partial deployment. In these examples, a physician, using the stent delivery catheter system 10, may reconstrain the stent 122 back to its compressed form within the lumen of the outer tubular member 110 during a medical procedure prior to full deployment and expansion of the stent 122. An advantage of a bi-directional delivery system permitting reconstrainment of the stent 122 being that adjustments to the position of the stent 122 can be made while the catheter shaft 102 and endoscope are already in position, eliminating the need to retract the catheter shaft 102 from the endoscope, replace it to better the precision of the target site which may extend the length of the medical procedure and/or cause more trauma to the patient's tissue.

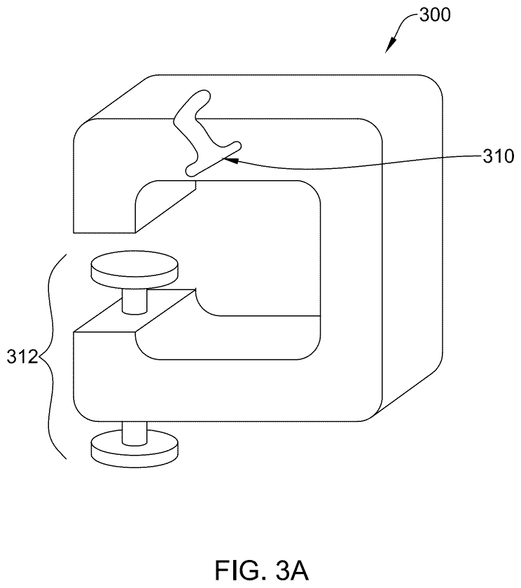

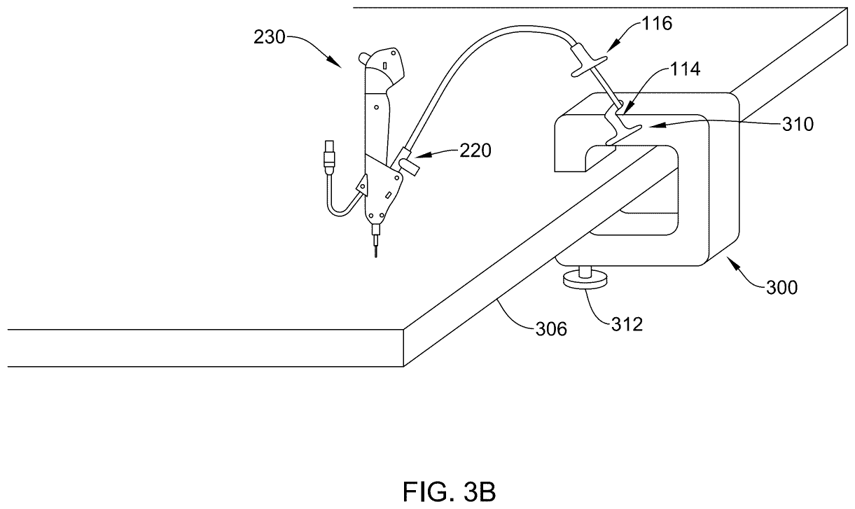

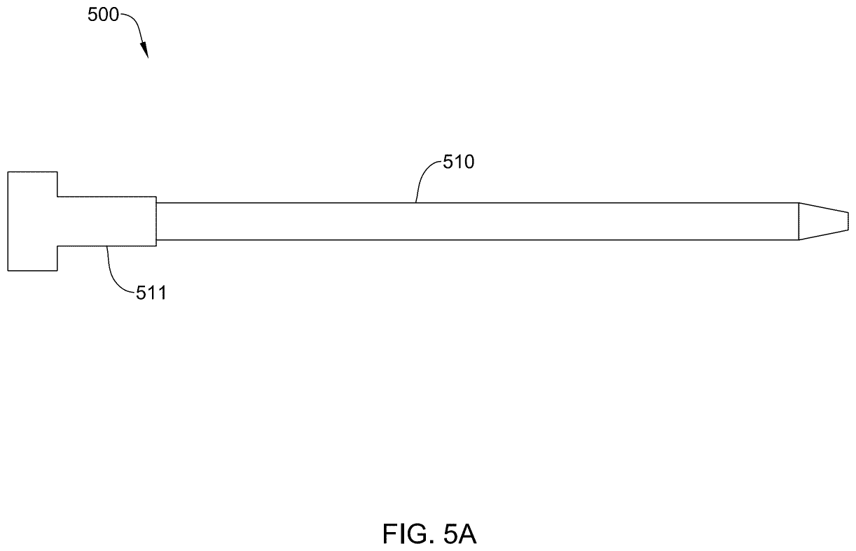

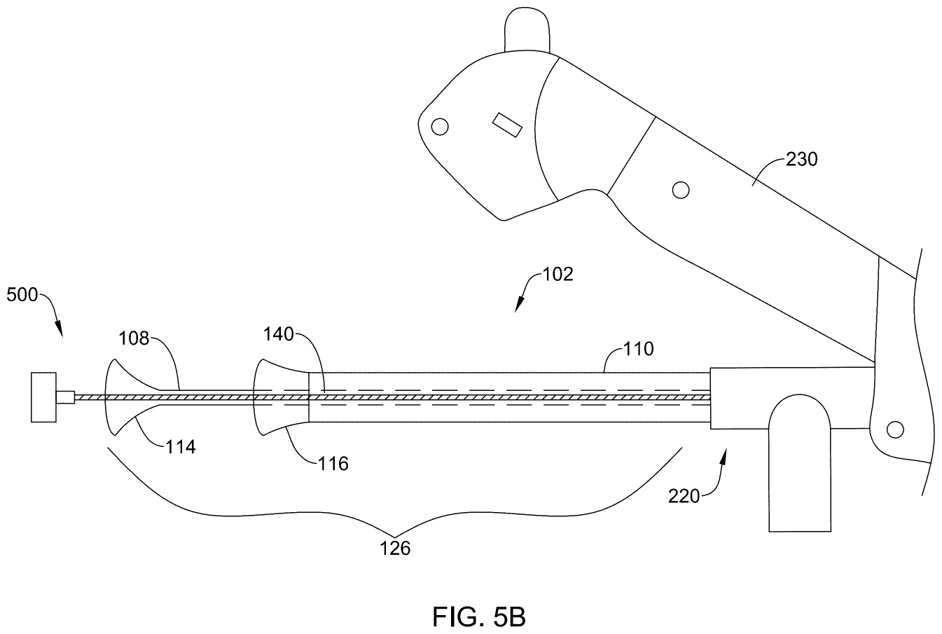

Thus, to aid in accurate deployment, the stent delivery catheter system 10 may be accompanied by an ancillary device to prevent unintended movement proximal of and exterior of the endoscope as illustrated in FIG. 2A, FIG. 3A, and FIG. 5A.

FIG. 2A and FIG. 2B illustrate a loop track ancillary device 200. Loop track ancillary device 200 comprises a continuous loop body 211 having a proximal end 217, a distal end 216, and a channel 210 along the length of the loop body 211 to form a loop track 215. The channel 210, and thus the loop track 215, may extend the entire length of the loop body 211 from the proximal end 217 to the distal end 216 and may open out laterally through a slot in the sidewall of the continuous loop body 211. The channel 210, and thus the loop track 215, may be sized and configured to receive the elongate shaft 102 of the stent delivery catheter system 10 therein. For example, the proximal portion 126 of the elongate shaft 102 remaining exterior of an endoscope may be positioned in the loop track 215 via the channel 210 (e.g.., laterally inserted into the loop track 215 via the slot in the sidewall of the continuous loop body 211).

The loop body 211 defining the loop track 215 may be generally arcuate and/or have an arced region 212 in which the loop body 211, and thus the loop track 215, follows an arcuate path. In some instance the arced region 212 may curve through an arc of 180 degrees or more, 270 degrees or more, or 300 degrees or more. In some instances, the arced region 212 may curve such that the loop body 211, and thus the loop track 215, includes a crossing point 213 in which the loop body 211, and thus the loop track 215, crosses over itself at a crossover location, and may resemble a Greek letter alpha (α). In other instances, the opposing end portions of the loop body 211 extending from the arced region 212 may converge toward one another without crossing one another, and may resemble a Greek letter omega (Ω).

In some embodiments, the channel 210 is configured to be of a uniform width from the proximal end 217 of the loop track 215 to the distal end 216 of the loop track 215. In other embodiments, the width of opening 210 may vary along its length to allow, for example, for variation in the diameter of the catheter shaft 102 or in the length of the catheter shaft 102 extending from the working channel of the endoscope, or to allow for accommodation of multiple catheters. In some embodiments, the loop body 211 is comprised of semi-rigid material to allow for some expansion of the channel 210 during operation, yet the loop body 211 is able to maintain is arcuate shape when not subject to external forces. The channel 210 may open out to, or be located along an inner radius of the arced region 212 (i.e., along the concave curved side of the arced region 212). In other instances, the channel 210 may be otherwise arranged along the loop body 211. In some embodiments, visual indicators may be included to define various default or optimized sizes of the adjustable loop track ancillary device 200.

The size (e.g., length) of the loop body 211 and the loop track 215 may be chosen in accordance to the needs of the endoscopy procedure, the attached stent delivery system, and the operational setting, among other factors. In some embodiments, the loop body 211 includes a mechanism, such as a telescoping mechanism, locking tabs, a ratchet and pawl mechanism, flexible plastic inserts, or flexible rubber inserts to vary the size (e.g., length) of the loop track 215 and/or the curvature of the arced region 212 prior to stent deployment.

In this embodiment, the proximal portion 126 of the catheter shaft 102 (the portion of the catheter shaft 102 extending proximal of the access port 220 of the endoscope 230 is placed and sits into the loop track 215 of the loop track ancillary device 200 to prevent movement of catheter shaft 102, as illustrated in FIG. 2C. The distal end 216 of the loop track ancillary device 200 is engaged with (e.g., coupled to, in abutment with) the access port 220 leading into the working channel of the endoscope 230. Some embodiments may include additional mechanisms to secure distal end 216 of the loop track 215 to the access port 220 leading into the working channel of the endoscope 230. In some embodiments, the loop track ancillary device 200 may also include visual alignment indicators to ensure proper alignment between the loop track ancillary device 200 and the access port 220 of the endoscope 230.

The orientation of the loop track ancillary device 200 relative to the working channel of the endoscope 130 may vary. With the catheter shaft 102 positioned within the loop track 215 of the loop track ancillary device 200 and the loop body 211 engaged with the access port 220 of the endoscope 230, the user may actuate the distal handle 116 toward the proximal handle 114 to proximally withdraw the outer tubular shaft 110 relative to the stent 122 and inner shaft 108 to deploy the stent 122. Since the loop track ancillary device 200 retains the curved configuration of the proximal portion 126 of the catheter shaft 102 stationary (e.g., doesn't allow the curvature of the proximal portion 126 of the catheter shaft to change due to lateral movement thereof), inadvertent movement of the inner shaft 108 is reduced.

FIG. 3A illustrates a clamp ancillary device 300. Clamp ancillary device 300 comprises a handle retention member 310 and a clamping mechanism 312. The clamp ancillary device 300 is configured to securely hold the proximal handle 114 of the stent delivery catheter system 10 stationary during stent deployment so as to prevent proximal handle 114 movement.

The handle retention member 310 is configured to receive the proximal handle 114 to secure the proximal handle 114 to the clamp ancillary device 300. In some embodiments, and as illustrated, the handle retention member 310 is a cavity, such as a push-fit slot, configured to receive the proximal handle 114 without any additional tools, only requiring the proximal handle 114 to be inserted into the cavity (e.g., snapped into place within the cavity). The shape of the cavity forming the handle retention member 310 may be similar to the shape of the proximal handle 114 such that the proximal handle 114 closely mates within the cavity. In some embodiments, the handle retention member 310 may also include additional features, such as ridges, clasps, or latches, to further secure the proximal handle 114 within the cavity. In some embodiments, the orientation of the handle retention member 310 (e.g., the cavity) may be configured to be adjusted in angle in order to, for example, accommodate varying orientations of the proximal handle 114, accommodate for different workspaces, enhance comfort and ergonomics during operation, better align the proximal handle 114 for user operation, or otherwise increase flexibility and precision in use of the clamp ancillary device 300. In some embodiments, clamp ancillary device 300 is manufactured as one piece, but in other embodiments, the clamp ancillary device 300 may be constructed out of multiple parts coupled together. For example, in some instances the portion of the clamp ancillary device 300 including the handle retention member 310 (e.g., cavity) provided with a first component of the clamp ancillary device 300 and the clamping mechanism 312 may be provided with a second component of the clamp ancillary device 300, in which the first component may be articulatable relative to the second component (e.g., may be pivotably coupled together) to adjust the position of the handle retention member 310 (e.g., cavity) relative to the clamping mechanism 312.

Clamping mechanism 312 securely attaches the clamp ancillary device 300 to a rigid support structure 306, for example, a table, a cart, a rail, a medical device console, or other stationary structure, so as to keep the clamp ancillary device 300 and the catheter shaft 102 steady. In some embodiments, the clamping mechanism 312 may be a C-clamp (illustrated), G-clamp, F-clamp, spring-loaded toggle clamp, bench clamp, spring clamp, pipe clamp, a quick-grip clamp (one-handed clamp), a pony clamp, a hand screw clamp, a vise grip (locking clamp), an edge clamp, a cam clamp, a screw clamp, a vacuum clamp, a suction clamp, a T-bar clamp, a rail clamp, a track clamp, a hold-down clamp, a pneumatic clamp, or a corner clamp. Some embodiments include a plurality of clamps, including a plurality comprising of disparate types of clamps, in order to provide more security or to provide ready-options for the user to choose from within the same device. For example, a clamping mechanism 312 may comprise of both a C-clamp and a vise grip to provide the user the option to either clamp onto large flat surfaces, like tables, with the C-clamp, or to smaller rounded surfaces, such as the rounded portions of an endoscope tower or a piece of furniture, using the vise grip. The clamping mechanism 312 may be constructed from any suitable materials, including plastics, metals, or wood, selected according to, for example, preference and availability of the manufacturer or compatibility with nearby clamping surfaces.

FIG. 3B illustrates the clamp ancillary device 300 in operation during a medical procedure. When the proximal handle 114 of the stent delivery catheter system 10 is securely received by handle retention member 314 of the clamp ancillary device 300, the proximal handle 314 is held stationary during the proximal retraction of distal handle 116, thus preventing movement of proximal handle 114, and thus preventing axial (e.g., proximal and/or distal) movement of the inner shaft 108 and stent 122, which would lead to inadvertent or inaccurate stent deployment.

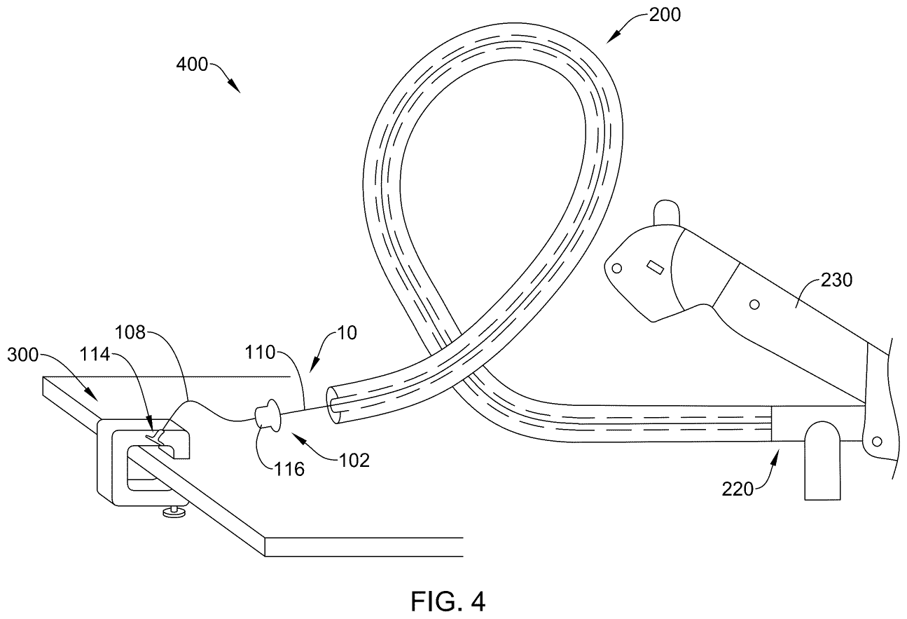

FIG. 4 illustrates the stent delivery system 10 advanced through the working channel of an endoscope 230, utilizing both loop track ancillary device 200 and the clamp ancillary device 300 used in conjunction with each other to form a combined ancillary device 400. Combined ancillary device 400 simultaneously inhibits movement of both the catheter shaft 102 via the loop track ancillary device 200 and movement of the proximal handle 114 via the clamp ancillary device 300, addressing both sources of the previously identified inadvertent movements. Simultaneous use of the loop track ancillary device 200 and the clamp ancillary device 300 provides the benefits of the individual components, which also synergize with each other to greatly eliminate sources of variation in deployment accuracy.

Operation and manufacture of each individual component of the combined ancillary device 400, the loop track ancillary device 200 and the clamp ancillary device 300, is the same as if they were used individually, as described above.

FIG. 5A illustrates a mandrel ancillary device 500. Mandrel ancillary device 500 includes a stiffening support shaft 510. In some embodiments, stiffening support shaft 510 is characterized as a mandrel.

Mandrel ancillary device 500 may be composed of any rigid material appropriate to constrain the proximal portion 126 of catheter shaft 102. For example, mandrel ancillary device 500 may be manufactured from stainless steel, a nickel-titanium alloy, such as nitinol, a nickel-chromium alloy, another metallic material, a polymeric material, or other material. In some embodiments, the mandrel ancillary device 500 may be manufactured using a semi-rigid material. With this construction, the mandrel ancillary device 500 has the ability to bend slightly once placed.

Relatedly, the length of the mandrel ancillary device 500 can vary in manufacturing in accordance with setting, preference, or character of mandrel ancillary device 500. In some examples, mandrel ancillary device 500 has a telescoping stiffening support shaft 510 to extend to a set of variety of lengths, or is otherwise configured to have an adjustable length.

In some embodiments, and as illustrated, mandrel ancillary device 500 further comprises of a handle 511 on the proximal end of the stiffening support shaft 510, configured to aid with loading the stiffening support shaft 510 into a lumen of the catheter 102. The stiffening support shaft 510 may extend distally from the handle 511 to a distal end of the stiffening support shaft 510. In some instances, the mandrel ancillary device 500 may have a tapered distal tip provided at the distal end of the stiffening support shaft 510.

FIG. 5B illustrates the mandrel ancillary device 500 in conjunction with the proximal portion 126 of the catheter shaft 102.

As shown in FIG. 5B, the stiffening support shaft 510 of the mandrel ancillary device 500 may be inserted into a lumen of the catheter shaft 102 (e.g., a guidewire lumen) and advanced through the proximal portion 126 of the catheter shaft 102. Mandrel ancillary device 500 is configured to be sufficiently rigid so as to straighten out all or part of the proximal portion of the catheter shaft 126, thus preventing bending and inadvertent movement of the stiffened proximal portion 126 of the catheter shaft 102.

The stiffening support shaft 510 of the mandrel ancillary device 500 may be advanced through the entire length, or substantially the entire length, of the proximal portion 126 of the catheter shaft 102 (the portion of the catheter shaft 102 extending proximally of the access port 220 of the endoscope 230) positioned exterior of the endoscope 230 to provide support to the proximal portion 126 of the catheter shaft 102. Accordingly, the stiffening support shaft 510 of the mandrel ancillary device 500 may be advanced through a lumen of the catheter shaft 102 until the distal tip of the stiffening support shaft 510 reaches the access port 220, extending into the access port 220, or extends into the working channel of the endoscope 230. Accordingly, the mandrel ancillary device 500 may be advanced through a lumen of the catheter shaft 102 until the distal tip of the stiffening support shaft 510 is positioned proximate or distal of the access port 220 of the endoscope 230.

In some embodiments, and as illustrated, the stiffening support shaft 510 is inserted into the guidewire lumen 140 of the catheter shaft 102 from an opening at the proximal end of the proximal handle 114 up to the working channel of the endoscope 230. If a guidewire already occupies at least a portion of the guidewire lumen 140, user may remove the guidewire to swap out the mandrel ancillary device 500 once the catheter shaft 102 has been advanced through the working channel of the endoscope to position the stent 122 at the target site. In a rapid-exchange configuration, the user may retain the guidewire position within the guidewire lumen, and rather, insert the stiffening support shaft 510 from an opening at the proximal end of the proximal handle 114 extending into the catheter shaft 102. In other embodiments, the stiffening support shaft 510 may be incorporated with the stent delivery system 10 such that that stiffening support shaft 510 may be selectively longitudinally movable within a lumen of the catheter shaft 102 depending on the length of the proximal portion 126 extending from the access port 220 of the endoscope 230. It should be appreciated, however, the mandrel ancillary device 500 may be inserted up to any point along the length of the catheter shaft 102 that the user feels is appropriate to the setting, procedure, or physical character of the catheter shaft 102 or mandrel ancillary device 500.

Once inserted into the catheter shaft 102, the presence of the stiffening support shaft 510 along the proximal portion 126 of the catheter shaft 102 provides support to the catheter shaft 102, precluding inadvertent movement and/or undesired lateral flexing of the catheter shaft 102 that may either prevent proximal retraction of the outer shaft 110 or create inadvertent force that would move the inner shaft 108 distally or proximally, and thus move the stent 122 relative to the desired deployment location is reduced.

In some instances, the inner shaft 108 and/or the outer shaft 110 may be sufficiently transparent or translucent such that the position of the distal end of the stiffening support shaft 510 may be visually observed through the catheter shaft 102 to ensure the stiffening support shaft 510 has been advanced a sufficient distance within the lumen of the stiffening support shaft 510. Visual indicators may also be included on the mandrel ancillary device 500 or the proximal portion 126 of the catheter shaft 102 to ensure consistent and optimized placement of the stiffening support shaft 510. For example, in some embodiments, visual indicators may be included to assist the user in verifying the proper depth of insertion of the stiffening support shaft 510 within the lumen of the catheter shaft 102. In other embodiments, visual indicators may also be included on the stiffening support shaft 510 to indicate the maximum insertion depth of the stiffening support shaft 510.

It should be understood that this disclosure is, in many respects, only illustrative. Changes may be made in details, particularly in matters of shape and size, without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A stent delivery system comprising:
a stent;
a catheter shaft comprising:
a distal portion and a proximal portion;
an inner shaft, the inner shaft comprising a proximal end and a distal end;
an outer tubular shaft having a proximal end and a distal end,
the outer tubular shaft slidably disposed over the inner shaft,
the outer tubular shaft configured to hold the stent in a constrained position proximate the distal end of the outer tubular shaft,
the stent configured to be deployed from the outer tubular shaft when the outer tubular shaft is retracted proximally relative to the stent;
a handle assembly at a proximal end of the catheter shaft, the handle assembly comprising:
a proximal handle coupled to the proximal end of the inner shaft; and
a distal handle coupled to the proximal end of the outer shaft,
wherein the outer shaft is configured to retract proximally together with proximal retraction of the distal handle while the proximal handle is held in a stable position to deploy the stent; and
an ancillary device comprising a loop body defining a loop track,
wherein the loop body has channel extending along a length of the loop body permitting the proximal portion of the catheter shaft to be laterally inserted into the loop track,
wherein the loop body is configured to constrain movement of the proximal portion of the catheter during deployment of the stent.

2. The stent delivery system of claim 1, wherein the loop body has an arced region extending in an arcuate path.

3. The stent delivery system of claim 2, wherein the arced region curves through an arc of 180 degrees or more.

4. The stent delivery system of claim 2, wherein the loop body crosses over itself at a crossover location.

5. The stent delivery system of claim 2, wherein opposing end portions of the loop body extending from the arced region converge toward one another without crossing one another.

6. The stent delivery system of claim 2, wherein the channel opens out to an inner radius of the arced region.

7. The stent delivery system of claim 1, wherein the channel extends an entire length of the loop body.

8. The stent delivery system of claim 1, wherein a distal end of the ancillary device is configured to engage an access port of a working channel of an endoscope.

9. The stent delivery system of claim 1, wherein the ancillary device further comprises a handle clamp configured to be coupled to the proximal handle to hold the proximal handle in a stable position.

10. The stent delivery system of claim 1, wherein the handle clamp comprises:
a cavity configured to receive the proximal handle and securely fit the proximal handle to the handle clamp; and
a clamping mechanism configured to clamp the handle clamp to a support structure.

11. A stent delivery system comprising
a stent;
a catheter shaft comprising:
a distal portion and a proximal portion;
an inner shaft, the inner shaft comprising a proximal end and a distal end;
an outer tubular shaft having a proximal end and a distal end,
the outer tubular shaft slidably disposed over the inner shaft,
the outer tubular shaft configured to hold the stent in a constrained position proximate the distal end of the outer tubular shaft,
the stent configured to be deployed from the outer tubular shaft when the outer tubular shaft is retracted proximally relative to the stent;
a handle assembly at a proximal end of the catheter shaft, the handle assembly comprising:
a proximal handle coupled to the proximal end of the inner shaft; and
a distal handle coupled to the proximal end of the outer shaft,
wherein the outer shaft is configured to retract proximally together with proximal retraction of the distal handle while the proximal handle is held in a stable position to deploy the stent; and
an ancillary device comprising a handle clamp configured to hold the proximal handle in a stable position, the handle clamp comprising:
a cavity configured to receive the proximal handle and securely fit the proximal handle to the handle clamp, and
a clamping mechanism configured to clamp the handle clamp to a support structure.

12. The stent delivery system of claim 11, wherein the cavity is configured to receive the proximal handle therein without use of any tools.

13. The stent delivery system of claim 11, wherein the ancillary device further comprises a loop body defining a curved loop track,
wherein the curved loop track opens out through a sidewall of the loop body along a length of the loop body, the curved loop track configured to receive the proximal portion of the catheter laterally therein through a slot in the sidewall of the loop body,
wherein the loop body is configured to constrain the proximal portion of the catheter shaft during deployment of the stent.

14. The stent delivery system of claim 13, wherein the loop body has an arced region extending in an arcuate path.

15. The stent delivery system of claim 14, wherein the arced region curves through an arc of 180 degrees or more.

16. The stent delivery system of claim 15, wherein the loop body crosses over itself at a crossover location.

17. The stent delivery system of claim 14, wherein opposing end portions of the loop body extending from the arced region converge toward one another without crossing one another.

18. A stent delivery system comprising
a stent;
a catheter shaft comprising:
a distal portion and a proximal portion;
an inner shaft, the inner shaft comprising a proximal end and a distal end;
an outer tubular shaft having a proximal end and a distal end,
the outer tubular shaft slidably disposed over the inner shaft,
the outer tubular shaft configured to hold the stent in a constrained position proximate the distal end of the outer tubular shaft,
the stent configured to be deployed from the outer tubular shaft when the outer tubular shaft is retracted proximally relative to the stent;
a handle assembly at a proximal end of the catheter shaft, the handle assembly comprising:
a proximal handle coupled to the proximal end of the inner shaft; and
a distal handle coupled to the proximal end of the outer shaft,
wherein the outer shaft is configured to retract proximally together with proximal retraction of the distal handle while the proximal handle is held in a stable position to deploy the stent; and
an ancillary device comprising a stiffening support shaft configured to be removably inserted into a lumen of the inner shaft through an opening at a proximal end of the proximal handle.

19. The stent delivery system of claim 18, wherein the stiffening support shaft has a length less than an entire length of the catheter shaft.

20. The stent delivery system of claim 18, wherein the stiffening support shaft includes at least one visual indicator.
